(19) United States
(12) Patent Application Publication
(10) Pub. No.: US 2010/0023021 A1
(43) Pub. Date: Jan. 28, 2010

(54) Biological Interface and Insertion

(76) Inventor: J. Christopher Flaherty, Topsfield, MA (US)

Correspondence Address: SoCAL IP LAW GROUP LLP, 310 N. WESTLAKE BLVD. STE 120, WESTLAKE VILLAGE, CA, 91362, US

(21) Appl. No.: 12/574,022

(22) Filed: Oct. 6, 2009

Related U.S. Application Data
(63) Continuation-in-part of application No. 11/319,547, filed on Dec. 29, 2005.
Application No. 11/319,547 is a continuation-in-part of application No. 11/316,807, filed on Dec. 27, 2005.
Application No. 11/316,807 is a continuation-in-part of application No. 11/320,709, filed on Dec. 30, 2005.
Application No. 11/320,709 is a continuation-in-part of application No. 11/321,860, filed on Dec. 30, 2005.

(51) Int. Cl. A61B 19/00

(52) U.S. Cl. 606/130

(57) ABSTRACT

Insertion systems and processes for inserting a medical implant are disclosed. An insertion system used to insert an implant into a patient's body may include a housing and a movable member configured to move relative to the housing. The system may further include an alignment detection mechanism disposed adjacent the housing and configured to detect an alignment information of the movable member with respect to at least one of the implant and a target site of the patient's body.

### RELATED APPLICATION INFORMATION

This patent claims priority from the following prior-filed copending non-provisional patent applications, which are incorporated herein by reference:

Application Ser. No. 11/319,547 filed Dec. 29, 2005;

Application Ser. No. 11/316,807 filed Dec. 27, 2005;

Application Ser. No. 11/320,709 filed Dec. 30, 2005; and

Application Ser. No. 11/321,860 filed Dec. 30, 2005.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

1. Field

This disclosure relates to apparatus and processes for inserting a medical implant, such as, for example, an electrode sensor, in a patient's body.

2. Description of the Related Art

Various parts of the body, such as, for example, the brain and sensory organs, generate signals (e.g., electrical signals) that contain information regarding an intended function or sensory state. To provide access to these electrical signals associated with numerous types of living cells in the patient's body, certain devices including one or more sensors may be implanted in various locations within the patient's body.

Accurate positioning of the sensor in the patient's body, however, may face many challenges. For example, since a target tissue to which a sensor or other implant is to be implanted may not have a flat or uniform surface, a proper alignment of the sensor with respect to the target tissue must be confirmed prior to the implantation of the sensor. This alignment confirmation process is extremely difficult and time consuming. Moreover, the confirmation process may not be highly accurate because, among other reasons, the surgeon typically uses visual inspection alone for the alignment confirmation.

Accurate positioning of the sensor or other implant may be even more difficult when the target tissue is constantly moving during the implantation procedure. For example, although a patient is typically placed under general anesthesia during an implantation procedure, certain parts of the body, such as, for example, the brain and various internal organs, may continue to move due to, for example, the patient's continued respiration and blood pressure (i.e., heart beat) functions. If the target tissue to which the sensor is to be implanted lies in one of those moving parts of the body, accurate positioning of the sensor or any other implant may be extremely difficult.

The continuous motion of the target tissue may not only cause inaccurate positioning, but may also lead to over-insertion and/or excessive insertion speed or impact force of the sensor at the tissue surface, resulting in excessive trauma of and/or damage to the target tissue. For example, over-insertion in the brain may result in sub- and epidural hemorrhage, spreading depression (e.g., transient depolarization of neurons), and potentially permanent brain cell damage. The excessive insertion speed may cause an excessive momentum transfer to the cortical tissue below the outer membranes (i.e., pia), resulting in tissue damage.

To avoid these potential problems, extreme care must be exercised by the surgeon during an implantation procedure. In particular, the surgeon may inspect the movement of the target tissue (e.g., often with naked eye) to properly time insertion of the sensor (e.g., when the target surface is at the top of its movement cycle). This process may result not only in an inaccurate positioning of the sensor, but also in a prolonged operation period (e.g., 2-5 hours to implant sensors).

### DESCRIPTION OF THE DRAWINGS

FIG. 1 is a schematic illustration of an insertion system used to insert an implant into a patient's brain.

FIG. 2 is a schematic cross-sectional view of an elongated housing of the system of FIG. 1, illustrating various components of the system.

FIG. 3 is a partial side view of a distal end portion of the elongated housing of FIG. 2, illustrating an alignment detection mechanism.

FIG. 4 is a bottom view of the elongated housing of FIG. 2, illustrating an exemplary arrangement of the alignment detection mechanism.

FIG. 5 is a bottom view of the elongated housing of FIG. 2, illustrating another exemplary arrangement of the alignment detection mechanism.

FIG. 6 is a bottom view of the elongated housing of FIG. 2, illustrating still another exemplary arrangement of the alignment detection mechanism.

FIG. 7-10 are schematic illustrations of the implant inserted into a target site.

FIG. 11 is a schematic illustration of an acceleration profile of a piston of the insertion system.

FIG. 12 is a schematic diagram illustrating operational steps of an insertion system.

Throughout this description, elements appearing in figures are assigned three-digit reference designators, where the most significant digit is the figure number and the two least significant digits are specific to the element. An element that is not described in conjunction with a figure may be presumed to have the same characteristics and function as a previously-described element having a reference designator with the same least significant digits.

### DETAILED DESCRIPTION

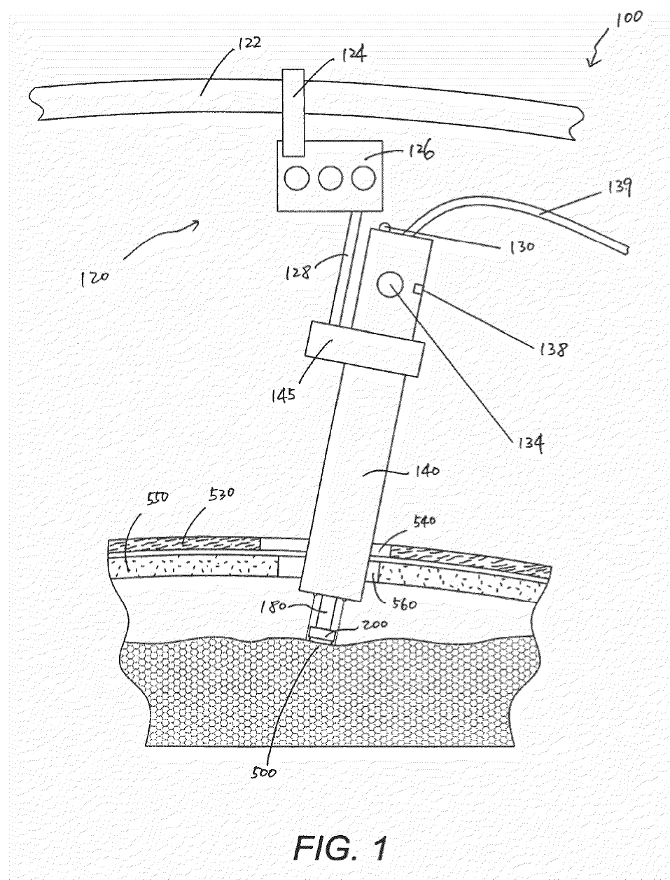

FIG. 1 shows an insertion system 100 used in, for example, inserting an implant 200 (e.g., two- or three-dimensional array of electrodes) into a patient's body, such as, for example, the brain (e.g., motor cortex of a human or animal brain). The system 100 may comprise an elongated housing 140 and a piston 180 coupled to the distal portion of the elongated housing 140. The housing 140 may have a length sufficient to reach the target site 500 in the patient's brain. The housing 140 may be coupled to an external device via a cable 139 for data communication, power supply, etc. The housing 140 may be inserted into openings 540, 560 formed through the scalp 530 and skull 550, respectively, of the patient. The piston 180 may be axially movable relative to the housing 140. As will be described in more detail herein, the system 100 may comprise an alignment detection mechanism for detecting topography of a target tissue site 500 to confirm proper alignment of the implant 200 relative to that tissue site 500 prior to insertion of the implant 200. The system 100 may also comprise a suitable control mechanism for controlling the movement of the elongated housing 140 and/or the piston 180.

The implant 200 may comprise a two- or three-dimensional electrode sensor having a plurality of projections extending from a base. At least one of the projections may include an electrode configured to detect electrical signals or impulses (e.g., electrical neural signals generated from neurons or other living cells) from the patient's body and may be arranged in an array, for example, in a 10.times.10, 8.times.8, or 5.times.5 matrix. The electrode may be positioned at the distal tip of the projection, however the electrodes may be positioned at any other position along the length of the projection. Some projections may include more than one electrode along their lengths. In various exemplary embodiments, the base of at least one of the projections may comprise a suitable tissue anchoring member, such as, a barbed projection.

The projections may have a variety of different types of electrodes or other functional elements, such as, for example, recording electrodes, stimulating electrodes, photo sensors, temperature sensors, pressure sensors, acoustic transducers, other physiological sensors known in the art, other transducers known in the art (e.g., light, heat, and magnetic transducers), or any combination thereof. The differences between these different types of electrodes or functional elements may include different materials of construction, coatings, thicknesses, lengths, geometric shapes, etc. In some exemplary embodiments, each of the recording electrodes may form a recording channel that may directly detect electrical signals generated from each of the neurons in the electrode's vicinity.

In one exemplary embodiment, one or more projections may comprise a photodiode for transmitting light (e.g., ultraviolet light) for stimulation of cells. In another exemplary embodiment, one or more projections may comprise a hollow space (e.g., a fluid reservoir) for storage and delivery of therapeutic agents or drugs. For example, arrays disclosed in application Ser. No. 10/717,924 by Donoghue et al., incorporated by reference, may be used. In still another exemplary embodiment, one or more projections may include a photodiode-transistor pair for transmitting light and detecting reflective light indicative of cellular signals, such as light transmitted at one or more wavelengths that is monitored for changes in those wavelengths in the detected light.

The insertion system 100 may be used to insert an electrode sensor 200 in various other locations in the patient's body, such as, for example, other parts of the central nervous system (e.g., spinal cord) or the peripheral nervous system (e.g., arms, legs, and muscles). In addition, the electrode sensor 200 may be inserted into an organ (e.g., heart, pancreas, kidney, liver, etc.) or tumor tissue (e.g., brain tumor or breast tumor), where the projections of the sensor 200 may penetrate deep into the desired tissue of the organ or tumor. Moreover, the system may be used to implant various other types of implantable medical devices, such as, for example, devices with transducers, miniaturized drug delivery assemblies (e.g., a chemotherapy device injected entirely into a tumor), a pacemaker or cardiac defibrillator lead, and a module including stem cells injected into the spine. In addition, the implant 200 may be placed above, partially below, or below the tissue surface of the patient's body.

In some exemplary embodiments, the devices with transducers may comprise: heat or cooling transducers for heat or cryogenic therapies; magnetic transducers for polarization or other magnetic therapies; light transducers for transmitting light to cause a drug activation or to induce a cellular response; sound and ultrasound transducers for imaging of tissue or impacting tissue; transducers that polarize cells or inject stimulation current.

Implantable devices may be for therapeutic purposes, diagnostic purposes, patient enhancement purposes, or any combination thereof. Potential therapeutic or diagnostic conditions may include, but not be limited to: obesity; an eating disorder; a neurological disorder such as epilepsy or Parkinson's Disease; a stroke; a coma; amnesia; irregular blood flow in the brain; a psychiatric disorder such as depression; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder such as stuttering; a physical injury; a migraine headache; and chronic or temporary pain.

As shown in FIG. 1, the insertion system 100 may comprise a frame 120 for supporting the elongated housing 140. The frame 120 may be a stereotactic frame, fixed relative to the patient's skull, that enable three-dimensional movement of the elongated housing 140 relative to the patient's body, so that the implant 200 may be accurately positioned with respect to the desired target site 500 of the patient's body. Alternatively, the frame 120 may be a fixed frame that may provide only a one- or two-dimensional degree of freedom. In an exemplary embodiment, the frame 120 may be mounted to the patient's body (e.g., head) near the target site 500 so that the frame may provide a consistent, stable frame of reference with respect to the target site 500. In an alternative embodiment, the frame 120 may be mounted to a bed frame or any other suitable structure in the surgical arena.

The frame 120 may comprise a movement controller 126 configured to control the movement of the housing 140 relative to the target site 500. The controller 126 may be coupled to the main frame 122 via a first arm 124. The first arm 124 may be slidably movable relative to the main frame 122 so that the controller 126, together with the elongated housing 140, may be horizontally displaced along a horizontal plane of the main frame 122. Alternatively or additionally, the first arm 124 may be fixedly coupled to the main frame 122, and the main frame 122 may be movable relative to another frame structure (not shown). The frame 120 may include a variety of mechanical and electromechanical devices, such as, cams, springs, linear actuators, stepper motors, servos, and solenoids, to control the movement of the housing 140.

The controller 126 may be coupled to the housing 140 via a second arm 128. The housing 140 may have a mounting member 145 coupled to the second arm 128. At least one of the controller 126 and the mounting member 145 is pivotably and/or rotatably coupled to the second arm 128, so that the housing 140 may vary its angular orientation with respect to the target site 500, as shown in FIG. 1. In addition, the second arm 128 may be axially movable relative to the controller 126 or the mounting member 145 of the housing 140. Alternatively, the mounting member 145 may be axially (e.g., slidably) movable along the longitudinal axis of the housing 140.

In some exemplary embodiments, the controller 126 may comprise a drive motor that may cause movement of the first and second arms 124, 128 or the mounting member 145 so as to control the movement of the housing 140 relative to the tissue site 500. For actuation of such movements, the controller 126 may receive controlling signals from an operator (e.g., surgeon or technician). Alternatively, the controller 126 may receive signals directly from the alignment detection mechanism, as will be described further herein, so that the controller 126 may automatically adjust the orientation and/or position of the housing 140, substantially eliminating the operator intervention.

According to some exemplary embodiments, the system 100 may not include the controller 126. In those embodiments, the housing 140 may be coupled to the frame 120, and its movement along the frame 120 may be controlled manually by the operator. In another exemplary embodiment, the system 100 may be operated without any frame 120. In this particular embodiment, the operator may hold and operate the housing 140 manually.

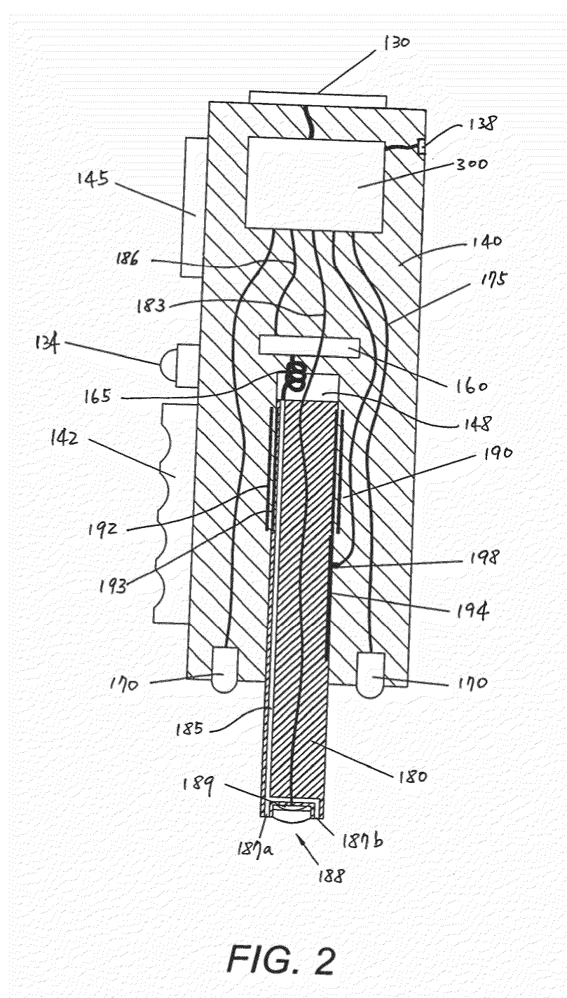

FIG. 2 schematically illustrates various components of the system 100 in more detail. The system 100 shown in this figure is different from the embodiment shown in FIG. 1, in that the system 100 is configured to be held by an operator. For that purpose, the housing 140 may comprise an operator grip 142 disposed on an outer surface of the housing 140. The grip 142 may be ergonomically designed (e.g., for left- or right-handed person) to facilitate holding of the housing 140 and/or to properly position cable 139, indicator 130, etc. In addition, the finger grip 142 may be configured such that the housing 140 may be aligned in a particular orientation when the operator places his/her fingers on the grip 142. In an exemplary embodiment, at least a portion of the housing 140 may be curved or bent so as to facilitate positioning of the housing 140 relative to the target site 500.

In various exemplary embodiments, the system 100 may comprise an alignment detection mechanism that may detect surface topography of the target site 500 to confirm proper alignment of the implant 200 relative to the target site 500 prior to or during insertion of the implant 200. The system 100 may also include an alignment status indicator 130 to provide the alignment information to the operator. In an exemplary embodiment, the alignment status indicator may include a signal light 130. When the alignment detection mechanism confirms proper alignment of the implant relative to the target site 500, the signal light 130 may be turned on to indicate the proper alignment. Other indication methods may also be used alternatively or additionally. For example, the signal light 130 may change in color or change from a blinking state to a continuously-lit state. The operator may then initiate insertion by, for example, actuating an actuation switch (e.g., pressing a button 134). In some exemplary embodiments, the actuation switch 134 may be positioned on or near the operator grip 142.

In an exemplary embodiment, the actuation switch 134 may be disabled until proper alignment of the implant 200 is confirmed by the alignment detection mechanism. Alternatively, the system 100 may be configured such that, while actuation of the actuation switch 134 is permitted to initiate the insertion process, the actual insertion of the implant 200 is delayed until the proper alignment of the implant 200 is confirmed by the alignment detection mechanism. In this particular embodiment, when the actuation switch 134 is actuated, the system 100 may automatically find the proper timing and alignment for insertion and automatically insert the implant 200 into the target site 500.

Alternatively or additionally, the alignment status indicator 130 may comprise a suitable display device (e.g., LCD screen) to provide more detailed alignment information to the operator. The information may be used by an operator to determine whether sufficient alignment is achieved to initiate the insertion. As briefly mentioned above, the alignment information may be directly fed to the controller 126 or other suitable processing device to automatically adjust the position/orientation of the housing 140 to a desired alignment condition. In an exemplary embodiment, the alignment information, including acceptable levels of misalignment, may be entered into the device by an operator (e.g., a clinician).

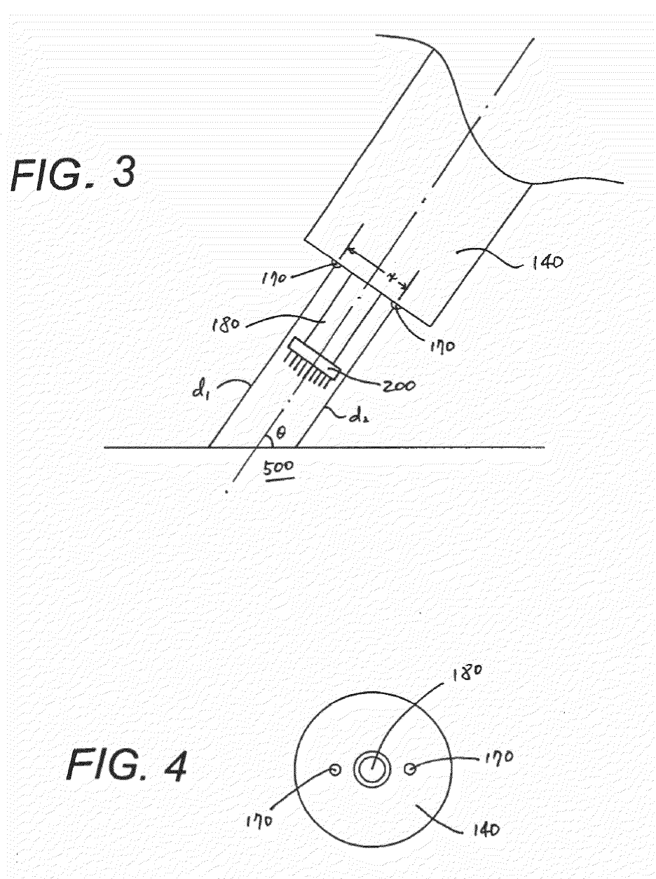

As best shown in FIGS. 2, 3, and 4, the alignment detection mechanism may comprise a photodiode/phototransistor pair 170 disposed at a distal end portion of the housing 140. The photodiode/phototransistor pair 170 may be connected to the module 300 via a suitable connection 175 for transmission of data and control signals, for example. As shown in FIG. 4, the photodiode/phototransistor pair 170 may be arranged symmetrically with respect to the piston 180. The photodiode/phototransistor pair 170 may be configured to measure reflection of light from the surface of the target site 500 to determine the distance d.sub.1, d.sub.2 from the tissue site 500 and, thereby, the angular alignment 0 of the implant 200 relative to the tissue site 500. For example, as shown in FIG. 3, each of the photodiode/phototransistor pair 170 may measure its distance d.sub.1, d.sub.2 from the tissue surface 500. The difference (d>−d.sub.2) in the measured distances may be used to determine the angular alignment (.theta.=tan−1.times. x d 1−d 2) of the implant 200 relative to the tissue surface 500, where x is a distance between the photodiode/phototransistor pairs 170. The photodiode/phototransistor pair 170 may be combined with a laser, one or more lenses (e.g., focusing lens, filter lens, etc.), or any other well-known optical techniques to facilitate the topography detection and/or distance measurement.

Various other types of detection mechanisms may be used alternatively or additionally. For example, radar may be used to send signals to the tissue surface 500 and measure the return signals (e.g., timing, signal strength, etc.) indicative of the topography of target site 500.

In some exemplary embodiments, the alignment detection mechanism may comprise a sound/ultrasound imaging device. The imaging device may comprise a directional transmitter for transmitting sound/ultrasound waves onto the surface of the target site 500 and a directional receiver for measuring the reflected waves to measure the acoustic timing (reflected signal) changes so as to determine the topography of the surface 500. In addition, the imaging device may utilize Doppler techniques to detect the motion of the target surface 500 by measuring frequency changes of the reflected signals. The Doppler techniques may be useful for application in the brain or other various internal organs since those parts of the body may be continuously moving during the insertion process. As will be described further herein, the functional module 300 or other suitable processing unit may analyze the motion of the target surface 500 to find the proper timing for implant insertion, such as timing determined based on information input by a clinician or other operator of the system.

According to another exemplary embodiment, the alignment detection mechanism may comprise a camera with a plurality of lenses to record the surface of the target site 500. The module 300 may comprise a three-dimensional image processing software to convert the recorded images to a three-dimensional map of the topography of the surface.

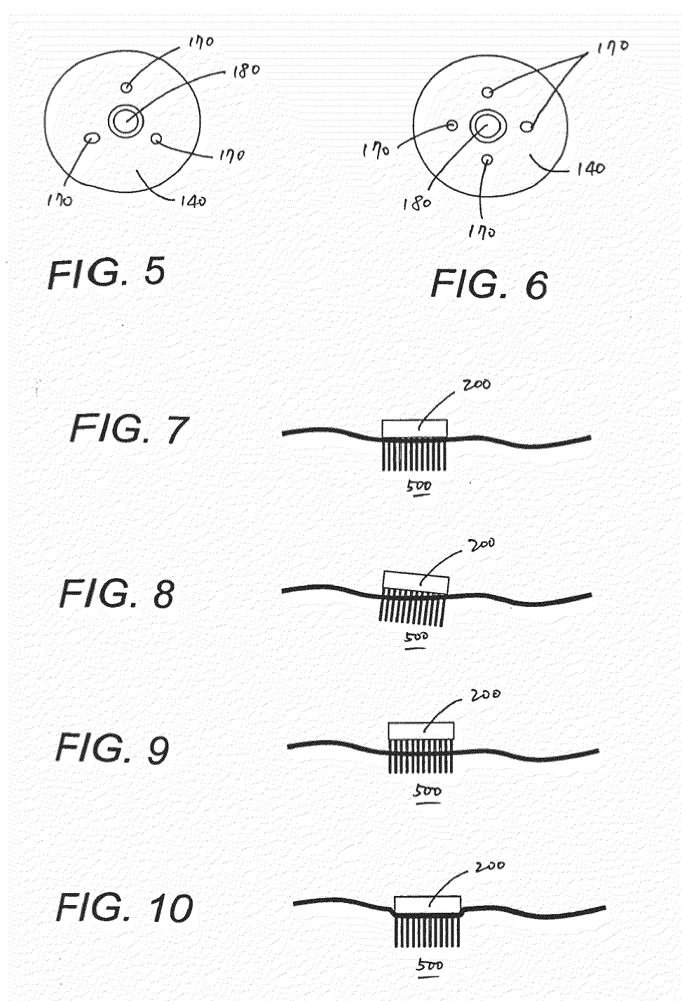

In some exemplary embodiments, as shown in FIGS. 5 and 6, the alignment detection mechanism may comprise three or more of any of the exemplary detection devices 170 discussed above. Multiple detecting devices 170 may enable more accurate measurement of the target surface 500 in a two- or three-dimensional space. In still another exemplary embodiment, the alignment detection mechanism may include a rotational or laterally-sweeping detector for a two- or three-dimensional measurement of the tissue surface.

The module 300 may be configured to receive input information relating to the desired alignment of the implant 200 relative to the target site 500. The input information may include, but not be limited to: maximum insertion force; depth of penetration; type or model of the implant being inserted; and any combination thereof. For that purpose, the module 300 may be connected to an external device (not shown), such as, for example, a computer or a PDA, via a programming port 138. Alternatively or additionally, the module 300 may include a wireless transceiver for transfer of information. Alternatively or additionally, the module 300 may include a suitable input device, such as, for example, a key pad or a touch screen (not shown). The input information may be adjustable or modifiable at any stage. For safety reasons, the external device or the input device may be password-protected, and only qualified individuals may access the input information. The external device may include different sets of personalized information for different patient types and different operator preferences.

The alignment information may include a specific target angle, or acceptable tolerance of the target angle, between the bottom surface of the implant 200 (or the bottom surface of the piston 180) and the surface of the target tissue 500. For example, in an exemplary embodiment, it may be desirable to have the bottom surface of the implant 200 and/or the piston 180 aligned substantially in parallel with respect to the surface of the target site 500, as shown in FIG. 7. In this particular embodiment, the alignment information may sufficiently prevent undesirable insertion conditions, such as, for example, insertion at off-angle (as shown in FIG. 8), under-insertion (as shown in FIG. 9), or over-insertion (as shown in FIG. 10).

In certain applications, however, those insertion conditions depicted in FIGS. 8-10 may be desirable. For example, in an exemplary embodiment, it may be desirable to have a predetermined angle between the bottom surface of the implant 200 and the surface of the target site 500, as shown in FIG. 8. Additionally or alternatively, it may be desirable to under-insert, as shown in FIG. 9, or over-insert, as shown in FIG. 10, the implant 200.

Other types of the alignment information may include, but not be limited to: a distance between the bottom surface of the implant 200 and the surface of the target tissue 500; velocity or acceleration of insertion (e.g., non-linear piston velocity, as will be described in more detail later); force of insertion or other force information; and information relating to desired under-insertion or over-insertion of the implant 200.

The module 300 may comprise a suitable processor configured to process the measured information to determine the topography of the target surface 500 and the alignment status of the implant 200 with respect to the target surface 500. The module 300 may then compare the detected alignment information (e.g., angular orientation and/or the distance) with the input information previously entered to determine the alignment status. The module 300 may have an acceptable tolerance range of values specified. In an exemplary embodiment, this tolerance range of values may be adjustable by an operator, such as a clinician.

The alignment information may then be fed back to the operator via the status indicator 130 to, if properly aligned, actuate the insertion or, if not properly aligned, manually stop the insertion and/or adjust the orientation of the housing 140 for proper alignment. Alternatively or additionally, the module 300 may transmit the alignment information to an appropriate component (e.g., the movement controller 126) of the system 100 or directly generate suitable control signals for the housing 140 or the piston 180 to automatically stop or prevent improper insertion of the implant 200 and adjust for proper alignment. For example, when the alignment mechanism detects improper alignment of the implant 200, the movement of the piston 180 may be automatically stopped, and the orientation of the housing 140 and/or the piston 180 may be adjusted according to the detected alignment information.

Referring to FIG. 2, the housing 140 may define an elongated internal space 148 for receiving the piston 180 therein. The piston 180 may be linearly movable along the longitudinal axis of the internal space 148 by a suitable linear drive mechanism. The linear drive mechanism may be controlled by the module 300 according to the desired input information preprogrammed into the module 300. For example, once the proper alignment is confirmed by the alignment detection mechanism and the insertion process is actuated, the movement of the piston 180 may be precisely controlled according to the input information (e.g., speed, acceleration, and/or force) stored in the module 300.

For example, the insertion process may be a closed-loop process where, during insertion, one or more parameters (e.g., velocity, acceleration, force, momentum, etc.) may be monitored, which may be compared with the prescribed input information. If the difference between the monitored value and the input information exceeds a predetermined threshold value, the insertion process may be stopped or adjusted to match the condition prescribed by the input information.

In some exemplary embodiments, the system may include a braking or deceleration mechanism that is configured to stop or decelerate the movement of the piston 180 or to control the movement of the piston 180 according to a specific deceleration profile. For example, the braking or deceleration mechanism may comprise an electromagnet assembly which can apply braking or deceleration forces to the piston 180. Alternatively or additionally, the braking or deceleration mechanism may comprise a controllable diameter friction collar arranged about the piston 180 to apply a braking force to the piston 180. The degree of braking or deceleration provided may be automatically adjusted.

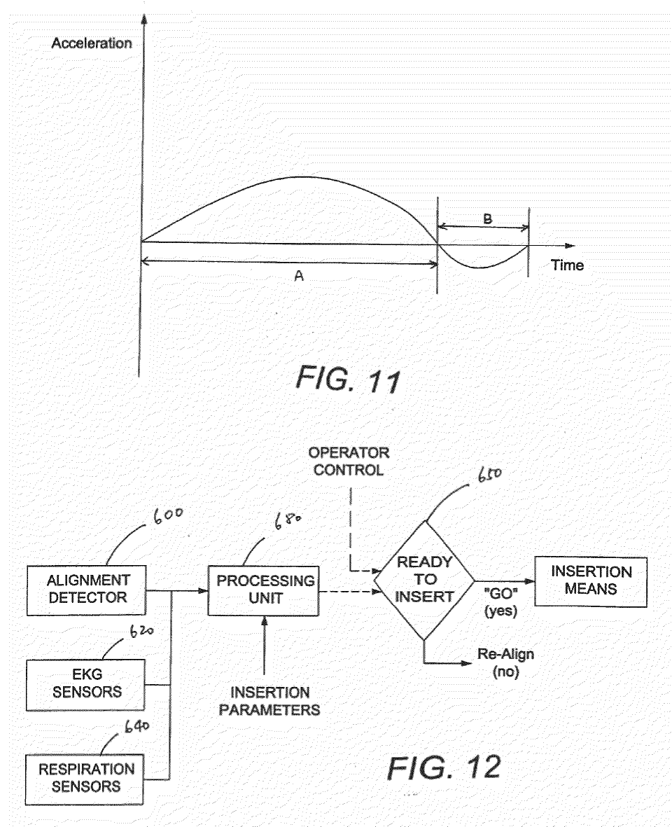

According to an exemplary embodiment, FIG. 11 schematically illustrates an exemplary acceleration profile of the piston 180 with respect to time, employing an initial rapid acceleration in period A followed by a deceleration in period B. The reason for the non-constant acceleration profile is, among other reasons, to minimize the tissue trauma caused by excessive momentum transfer to the tissue. For example, while a certain level of momentum may be required to penetrate the outer membranes of the brain (e.g., pia), the cortical tissue underlying the membranes may not provide sufficient resistance to the insertion impact, potentially resulting in over-insertion and tissue damage. Therefore, to reduce the momentum transfer into the cortical tissue, the non-constant acceleration profile may be employed to enable the implant 200 to penetrate through the outer membrane at a high speed with a high momentum and, thereafter, slow down the insertion speed to keep the cortical tissue from absorbing the insertion momentum. Thus, period A may represent the time it takes for the implant 200 to penetrate the outer membrane, and period B may represent the remaining time or distance until the implant 200 is positioned in a desired location.

In some exemplary embodiments, the durations of period A and period B or the overall acceleration profile may be determined by a contact/force measurement sensor 188, as will be described further herein. Alternatively, the precise timing may be predetermined by the module 300 or preprogrammed by an operator. Alternatively, an external device may be used to determine the timing and to transmit the information to the module 300.

In an exemplary embodiment, the system is configured to insert a number of different types of implants, each of which may have different parameters for the insertion. For that purpose, the module 300 may receive an input information (e.g., model number) via an input device so that the system may set or adjust its parameters to accommodate the type of implant being inserted.

The acceleration profile shown in FIG. 11 is exemplary only and any other acceleration or velocity profile may be used instead. For example, the piston 180 may move at a substantially constant velocity for a substantial portion of its travel. In another exemplary embodiment, the movement of the piston 180 may be stopped immediately before the implant is inserted at a desired final position. Even though the piston 180 is stopped, the momentum of the implant may cause the implant to continue its travel to the final position. This feature may be useful when the piston does not hold the implant.

As shown in FIG. 2, an exemplary embodiment utilizes an electro-magnetic drive mechanism 190. The mechanism 190 comprises a series of magnets 192 disposed within the housing 140 and along at least a portion of the internal space 148, and one or more electromagnets 193 disposed on a portion of the piston 180. The electromagnets 193 may be controllable by a suitable control circuit in the module 300. Alternatively, the piston 180 may include the magnets 192, and the housing 140 or the internal space 148 may comprise the one or more electromagnets 193. In operation, suitable current is selectively applied to one or more electromagnets 193 to create magnetic fields that may react with the magnetic fields of the magnets 192 and, thereby, cause the piston 180 to advance or retract, as well as start and stop motion, in a highly precise manner.

Alternatively or additionally, the system 100 may comprise any other suitable linear drive mechanism. For example, various exemplary embodiments of the linear drive mechanism may include, but not be limited to: a pneumatic drive assembly; a stepper motor associated with a lead screw; a pinch roller positioned in a fixed location within the internal space 148 in contact with a surface of the piston 180; a gas discharge/suction mechanism associated with the piston 180; a hydraulic or pneumatic piston drive mechanism utilizing a telescopic piston; an inch-worm drive mechanism; or any other drive mechanism known in the art.

The system 100 may also comprise a position indicator for continuously monitoring the position of the piston 180 (e.g., the position of the distal end of the piston relative to the target tissue 500). The positional information may be fed back to the module 300 or an external device to determine/monitor the precise motion of the piston 180 (e.g., velocity, acceleration, force, etc.). The positional information, in combination with the information regarding target tissue location, may also be used to determine the distance required for the piston 180 to travel for proper insertion of the implant 200 at the desired tissue depth.

In the exemplary embodiment shown in FIG. 2, the position indicator device may comprise a resistive strip 194 associated with a wiper 198 to measure a position and/or displacement of the piston 180. For example, in an exemplary embodiment, one end of the resistive strip 194 is connected to a first electrical wire, and the wiper 198 is connected to a second electrical wire. The wiper 198 may travel, coincident with the travel of the piston 180, from a first end of the resistive strip 194 to a second end of the resistive strip 194. When the wiper 198 is close to the first end, the resistance between the first and second wires is low. As the wiper 198 moves away from the first end, the resistance increases. The resistance may then be correlated to the position and/or displacement of the piston 180. The resistive strip 194 may be manufactured to be relatively linear, logarithmic, etc. Any other types of resistive strip 194 known in the potentiometer art, such as linear encoders and linear potentiometers, may be used alternatively or additionally.

The distal end of the piston 180 may be configured to releasably hold the implant 200 prior to insertion, as shown in FIG. 1. In some exemplary embodiments, the piston 180 may not hold the implant. For example, the implant 200 may be placed above or on a target surface, and the piston 180 is aligned with respect to the target surface and the implant 200.

In the exemplary embodiment shown in FIG. 2, the system 100 utilizes a suction mechanism to hold the implant 200 at the distal end of the piston 180. The piston 180 may define a lumen 185 extending between its proximal and distal ends, and the proximal end of the lumen 185 may be connected to a suitable suction source, such as, for example, a vacuum generator 160, via a suitable suction line 165. At its distal end, the lumen 185 may connect to two or more openings 187a, 187b disposed in the peripheral region of the distal end of the piston 180. This configuration may result in more stabilized holding of the implant 200 onto the piston 180. Alternatively, the distal end of the piston 180 may define only one opening in the central region or an opening with a cross-sectional area greater than that of the lumen 185. In operation, to hold the implant 200 with the distal end of the piston 180, the vacuum generator 160 may be turned on to exert sufficient suction force against a surface of the implant 200. The surface of the implant 200 may have a textured, smooth, or slotted portion to mate with the distal end of the piston 180.

Once the implant 200 is properly inserted in the patient's body, the vacuum generator 160 may be turned off or the lumen 185 may be closed to release the implant 200 from the piston 180. Alternatively, the vacuum generator may release the implant 200 prior to the completion of the implant insertion (e.g., before the implant 200 is finally placed at a desired location). Any other suitable holding mechanisms, such as, for example, a magnetic or electromagnetic holding mechanism, a mechanical connector, or a frictional engagement surface, may be used additionally or alternatively. For example, the implant 200 may comprise a mating slot, pin, or cap, and the distal end of the piston 180 may have a corresponding structure for releasably mating with the slot, pin, or cap. In some exemplary embodiments, the release of the implant 200 may be automatically performed. For example, the holding mechanism may be controlled by the module 300 through a connection 186 such that the holding mechanism may automatically release the implant 200 in response to certain conditions, such as, for example, proper insertion of the implant 200 in the target tissue 500.

As mentioned above, the piston 180 may comprise a contact/force measurement sensor 188, such as a strain gauge or pressure transducer. The contact/force measurement sensor 188 may be connected to the module 300 via a suitable connector 183 for transmission of data and control signals, for example. In some exemplary embodiments, the contact/force measurement sensor 188 may comprise a piezo crystal that creates a current and/or voltage in response to a force placed upon the crystal and/or the resulting deformation. The resultant current and/or voltage may be correlated to a force and/or displacement. The contact/force measurement sensor 188 may continuously monitor the contact force (e.g., piston force) against the target tissue 500 as the implant 200 penetrates into the tissue. Based on the detected contact force, the insertion speed of the implant 200 may be varied along the depth of the target tissue 500 such as to minimize the trauma to the target tissue 500 or otherwise optimize the insertion process.

In some exemplary embodiments, the piston 180 may comprise a temperature sensor for detecting temperature of the target tissue during or prior to the insertion. The detected temperature information may be transmitted to the module 300 to be taken into account in adjusting the insertion or alignment of the implant 200. For example, during an open surgical procedure, the surface temperature of tissue may vary (e.g., cooler than 98.6 degrees F.), and the optimum insertion velocity may change. For example, cooler tissue may be more rigid and may allow insertion of implant at a slower insertion velocity. Insertion at a lower velocity may be preferred to minimize momentum transfer of the implant 200 to the tissue.

According to another exemplary embodiment, the system 100 may include a motion detector. As explained above, the motion detector may utilize Doppler techniques or other signal analysis techniques to analyze and characterize the motion of the target surface 500 so as to properly time the insertion. Since the motion of the target surface 500 is typically related to certain repetitive body functions, such as, respiration and heart beat movements, analyzing and characterizing the detected motion of the target surface 500 may generate identifiable timings or cycle for the insertion of the implant 200. The system 100 may also comprise a memory storage device (e.g., in the module 300) to store the information relating to previous insertions for use in future insertions or other information such as implant information parameters.

In some exemplary embodiments, the system 100 may comprise other physiological sensors, such as, for example, an electrocardiogram (EKG) sensor for monitoring the patient's heart beat, a pressure sensor for monitoring the patient's blood pressure, or a respiration sensor for monitoring the patient's respiration pattern. In these particular embodiments, as illustrated in FIG. 12, the heart beat detected by the EKG sensor 620 and the respiration pattern detected by the respiration sensor 640 may be transmitted to a processing unit 680, in which this information may be analyzed and processed with the information collected from the alignment detector 600. Various insertion parameters discussed above may be input into the processing unit 680 and may be stored in memory.

The processing unit 680 may then generate a collective signal 650 as to whether the system 100 is ready to insert the implant 200. The operator may intervene during this process. If the collective signal 650 indicates that the system 100 is ready (e.g., “GO”-signal), a suitable control signal may be transmitted to the linear drive mechanism via, for example, a piston actuator in the module 300, to actuate the motion of the piston 180. Alternatively or additionally, the operator may manually actuate the linear drive mechanism by, for example, pressing the actuation button 134. If, on the other hand, the collective signal 650 indicates that the system 100 is not ready, a suitable control signal may be transmitted to an appropriate component of the system 100, as discussed above, to adjust the system 100 for proper alignment.

In still another exemplary embodiment, the insertion system 100 may be a modular system that may be used with an external device (e.g., a central data storage device or a main computer). The external device may be commonly used with another insertion system. Alternatively or additionally, the insertion system 100 may comprise two or more discrete components, each of which may be used with various other external devices. For that purpose, the insertion system 100 and/or each of the discrete components of the system 100 may have a unique identifier and may be configured to record all activities it performs. The recorded information may be retrieved for use in future insertion processes.

Although the figures show that the alignment detection mechanism is integrated into the housing 140, in some exemplary embodiments, the alignment detection mechanism may be provided as a separate device from the housing 140. For example, the alignment detection mechanism may be mounted to a fixing device which is aligned with the housing 140, such as a separate device mounted to the same stereotactic frame that the housing 140 may be mounted to.

### CLOSING COMMENTS

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. An insertion system used to insert an implant into a patient's body, comprising:
a housing;
a movable member configured to hold the implant and to move in a non-linear acceleration profile relative to the housing during insertion of the implant;
an alignment detection mechanism comprising a signal light, the alignment detection mechanism integrally formed with the housing and configured to
detect an alignment information of the moveable member with respect to the implant and a target site of the patient's body.

2. The system of claim 1, wherein the housing defines an internal space and the movable member is disposed at least partially in the internal space.

3. The system of claim 2, wherein the internal space extends along a longitudinal axis and the movable member is movable along the longitudinal axis.

4. The system of claim 1, wherein the alignment detection mechanism is configured to detect topography of the target site.

5. The system of claim 1, wherein the alignment indicator is fixed to the housing.

6. The system of claim 1, wherein the alignment information comprises angular orientation of the movable member with respect to the target site.

7. The system of claim 1, wherein the acceleration profile comprises an initial acceleration during a predetermined period followed by a deceleration before the movable member stops.

8. The system of claim 1, wherein the movable member comprises an opening in fluid communication with a suction source for holding the implant.

9. The system of claim 1, further comprising an electronic module configured to press the alignment information detected by the alignment detection mechanism, wherein an electrocardiogram sensor for monitoring the patient's heart beat so as to determine motion of the target site, wherein the electronic module is configured to analyze the detected motion of the target site so as to determine a timing for insertion of the implant.

10. The system of claim 1, further comprising an electronic module configured to press the alignment information detected by the alignment detection mechanism, wherein a respiration sensor for monitoring the patient's respiration pattern so as to determine motion of the target site, wherein the electronic module is configured to analyze the detected motion of the target site so as to determine a timing for insertion of the implant.

11. A method of inserting an implant into a patient's body using an inserter, the inserter comprising: a housing; a movable member configured to move relative to the housing; and an alignment detection mechanism disposed adjacent the housing; the method comprising:
detecting a characteristic of a target surface so as to determine a proper timing for insertion of the implant
placing the implant on the movable member
placing the inserter near the target surface
detecting alignment of the movable member relative to the target surface with the alignment detection mechanism, including detecting a distance between the movable member and the tissue surface
upon confirming that the implant is in a desired position, inserting the implant into the patient's body by moving the movable member towards the target surface

12. The method of claim 11, wherein detecting alignment comprises detecting topography of the target surface.

13. The method of claim 11, wherein detecting a characteristic of the target surface comprises monitoring the patient's heart beat and characterizing the pattern of the heart beat with respect to time.

14. The method of claim 11, wherein detecting a characteristic of the target surface comprises monitoring the patient's respiration function and characterizing the pattern of the respiration with respect to time.

15. A method of inserting an implant into a patient's body using an inserter, the inserter comprising: a housing and a movable member configured to move relative to the housing; the method comprising:
placing the inserter near a target surface so that the movable member is positioned at a distance from the target surface
detecting a characteristic of the target surface by monitoring at least one of a motion of the target surface, the patient's heart beat, the patient's blood pressure, and the patient's respiration pattern
analyzing the detected characteristic of the target surface so as to determine a proper timing for insertion of the implant
inserting the implant into the patient's body during the proper timing by moving the movable member towards the target surface.

16. The method of claim 15, wherein detecting alignment comprises detecting a distance between the implant and the target surface.

17. The method of claim 15, wherein analyzing the detected characteristic of the target surface comprises characterizing the pattern of the heart beat with respect to time.

18. The method of claim 15, wherein analyzing the detected characteristic of the target surface comprises characterizing the pattern of the respiration with respect to time.
